(19) United States
(12) Patent Application Publication
(10) Pub. No.: US 2004/0248861 A1
(43) Pub. Date: Dec. 9, 2004

(54) Modified delta5-androstenes having improved bioavailability

(76) Inventor: Bruce W. Kneller, Indianapolis, IN (US)

Correspondence Address: Law Office of Steven B. Leavitt, 9914 Waterview Parkway, Rowlett, TX, 75089, US

(21) Appl. No.: 10/785,600

(22) Filed: Feb. 23, 2004

Related U.S. Application Data
(60) Provisional application No. 60/499,285, filed on Aug. 29, 2003.

(51) Int. Cl. A61K 31/56; A61K 31/58; A01N 45/00; C07J 1/00

(52) U.S. Cl. 514/169; 552/502

(57) ABSTRACT

The hormone 7-keto-dehydroepiandrosterone (7-keto-dehydroepiandrosterone; Δ5-androstene 3β-hydroxy, 17-one; referenced as “7-keto-DHEA”) is modified at the 3^{rd} carbon, the 17^{th} carbon, or at both 3^{rd} carbon and the 17^{th} carbons with one or more of the following: tetrahydropyranyl (both mono and di-ethers), 1-methoxycyclopentane (both mono and di-ethers), cyclopent-1′-enyl (both mono and di-ethers), or combinations thereof. This modification improves oral bioavailability and plasma half-life in humans and mammals.

### BACKGROUND OF THE INVENTION

1. Technical Field

The present invention relates to hormones that are modified to improve their efficacy. More particularly, the invention relates to Δ5-androstenes that are modified to improve and increase the oral bioavailability and plasma half-life in mammals.

2. Description of Related Art

The hormone DHEA is a naturally produced steroid in humans which is produced in the adrenals glands, testes, and brain. DHEA is an important hormone as it is further converted to generate other essential hormones such as androgens and estrogens. Because DHEA is converted into androgens (such as testosterone), supplemental DHEA treatment often leads to unwanted side effects including: balding, facial and body hair growth, breast growth or acne.

The hormone 7-keto-dehydroepiandrosterone (7-keto-dehydroepiandrosterone; Δ5-androstene 3β-hydroxy, 17-one; referenced as “7-keto-DHEA”) is a metabolite of dehydroepiandrosterone (DHEA)—i.e., it is made from DHEA by enzymes in the body). Unlike DHEA and other steroids, 7-keto-DHEA does not metabolize into estrogens or testosterone. Thus 7-keto-DHEA has no androgenic or estrogenic side effects and the hormonal side effects are absent.

Research indicates many likely benefits of 7-keto-DHEA include the following:

Fat reduction;

Increased Energy/Reduced fatigue;

Enhanced Memory;

Improved Immune System Function;

Improvement of muscularity in wasting syndrome by decreasing the body's catabolic (muscle destroying) processes;

Heart: increases HDL cholesterol, reduces risk of heart disease;

Skin: diminishes wrinkles; moisturizes skin;

Improved Mood;

Increased sex drive;

Anti-aging: reverses tissue “deterioration”, rejuvenates the body;

In patients with lupus, a decrease in lupus symptoms;

In patients with Diabetes: normalized blood sugar; and

Improvement of HIV-related symptoms.

While DHEA supplements may improve these, DHEA given in doses needed to produce results often results in the unwanted production of sex hormones leading to secondary adverse effects. Therefore, others in the prior art have modified DHEA to produce various substituted Δ5-androstenes for producing the desired effects without the unwanted sex hormones. Regardless, frequent dosing is needed when given as an oral supplement because much of the hormone is negated during its first pass through the liver. Therefore, improved supplements containing various effective substituted Δ5-androstenes are needed.

### SUMMARY OF THE INVENTION

The bioavailability and half-life have not been established for 7-keto-DHEA. As with most other steroidal hormones, micronization improves bioavailability. The presence of other substances that use the same metabolic enzymes can improve bioavailability or half-life, but can also cause side effects.

The present invention describes a method for improving of the bioavailability and half-life of 7-keto-DHEA. Frequent of the hormone is needed when given as an oral supplement because much of the hormone is negated during its first pass through the liver. Therefore, improved supplements containing various effective substituted Δ5-androstenes are needed.

### DETAILED DESCRIPTION

The hormone DHEA is a naturally produced steroid in humans which is produced in the adrenals glands, testes, and brain. DHEA is the most abundant circulating hormone in humans but circulating levels decline significantly in late adulthood. Circulating levels of will continue to decrease with age for unknown reasons. This phenomena has lead many to suggest that maintaining levels of DHEA may counteract many of the pathophysiological effects of aging. Because the FDA considers it a naturally occurring vitamin, the sale of DHEA is unregulated. It has been available over-the-counter as a nutritional supplement since the mid 1990s.

DHEA is an important hormone as it is further converted to generate other essential hormones such as androgens and estrogens. After being secreted by the adrenal glands, it circulates in the bloodstream as DHEA-sulfate (DHEAS) and is converted as needed into other hormones including testosterone and androstenedione, thus influencing practically every organ and tissue in the body, including the brain.

Adequate levels of DHEA in the blood stream are important for many hormonal and metabolic functions. These functions are believed to be related to weight control, immune response, neurological wellness and others.

Circulating levels of the hormone may be increased by administration of supplemental DHEA. In patients with adrenal deficiency, supplemental DHEA led to benefits including: increased alertness and stamina, improved sense of wellbeing and enhanced sexual interest and enjoyment. Others with normal levels of DHEA have also benefited from supplemental administration.

However, individuals with normal levels of DHEA who take high doses of DHEA are likely to experience unwanted side effects because the hormone is readily converted into androgens such as estrogen and testosterone. Patients (with normal adrenal function) who have taken supplemental DHEA have demonstrated the following side effects: hair loss, increased facial hair growth, acne (in over 50% of patients), increased perspiration and odor, scalp itching, menstrual irregularities, irritability and restlessness. Moreover, the long term effects of high levels of DHEA are largely unknown. And studies have produced data suggesting that high levels of DHEA may increase the risk of breast and prostate cancer. Other studies have correlated high levels of DHEA with increased blood pressure and other cardiovascular risk factors. DHEA has been modified to produce various Δ5-androstenes for producing the desired effects while minimizing side effects. The present invention relates to a modified form of the hormone, 7-keto-dehydroepiandrosterone.

The hormone 7-keto-dehydroepiandrosterone (7-keto-dehydroepiandrosterone; Δ5-androstene 3β-hydroxy, 17-one; referenced as “7-keto-DHEA”) is a metabolite of dehydroepiandrosterone (DHEA). In other words, it is made from DHEA by enzymes in the body. 7-keto-DHEA was discovered in the 1950s but received little research attention until the 1990s. Scientists are currently studying the steroid and related compounds for use in fat reduction, memory enhancement, immune system regulation, skin rejuvenation, dermatitis treatment, and wasting syndrome.

Unlike DHEA and other steroids, 7-keto-DHEA is not metabolized into estrogens or testosterone. Because 7-keto-DHEA of this, supplemental DHEA treatment often yields the desirable effects of DHEA treatment without the unwanted side effects. Thus 7-keto-DHEA has no androgenic or estrogenic side effects—the hormonal side effects are absent.

Benefits of Administration of 7-keto-DHEA

Research indicates many benefits of administration of 7-keto-DHEA without the unwanted side effects typical of other hormone treatments. Moreover, the hormone is naturally occurring and non-toxic.

Fat Reduction and Energy

7-keto-DHEA decreases the efficiency of energy production in the body—more fats have to be burned to produce the same amount of useable energy. This results in loss of fat (weight loss). If one runs low on fat then one would presumably experience a reduction in energy production. For fat loss, doses of 350 to 1400 mg/day are used. In recent studies, patients using 7-keto-DHEA as a supplement to diet demonstrated statistically significant decreased body weight and body fat as well as increased T3 thyroid stimulating hormone.

Memory

Experiments in mice have shown that a prohormone of keto-DHEA improves memory at doses of 20 mg/kg/day. Extrapolating these results to humans and keto-DHEA, in proportion to bodyweight, gives a dose of about 1400 mg/day.

Immunity

This claim is based on experiments with mice infected with flu viruses. 7-keto-DHEA treatment caused an increase in anti-viral antibodies to some of the viruses. However, dosages used were impossible to decipher from the data.

HIV and Muscle

Monkeys infected with a virus similar to HIV, and suffering from wasting syndrome, regained bodyweight when treated with 7-keto-DHEA at doses as low as 8 mg/kg/day (which extrapolates to 475 mg/day for a 130 lb human). Human studies, and studies on wasting conditions other than HIV, have not yet been done.

Heart

A Czech study showed that a single transdermal dose of 25 mg improved blood levels of HDL-cholesterol and other substances relevant to vascular and heart disease.

Skin

These claims are made in U.S. Pat. Nos. 6,399,084 and 6,399,085, but no supporting evidence is presented.

Lupus, Mood, Heart, Diabetes, Sex, Anti-aging

These claims are just repetitions of claims made for DHEA. There is no published data on 7-keto-DHEA that supports or refutes any of them.

Hormonal Effects

It is known that 7-keto-DHEA does not metabolize to estrogens or testosterone, and that supplementary 7-keto-DHEA can decrease testosterone and estradiol levels in the blood. It seems reasonable to suppose that this translates into a lack of estrogenic and androgenic side effects.

Salivation

In monkeys at very high doses (equivalent to about 70 grams/day for a human), 7-keto-DHEA caused excess salivation. No such side effect occurs at the doses one would actually use.

Moreover, other studies have provided evidence the 7-keto-DHEA may have other beneficial effects including:

As a diet supplement: promoting weight loss, decreasing body fat;

Energy: increases “energy”, reduces fatigue;

Muscle: improves muscularity in wasting syndrome by decreasing the body's catabolic (muscle destroying) processes;

Heart: increases HDL cholesterol, reduces risk of heart disease;

Lupus: decreases lupus symptoms;

Mood: reduces depression, improves mood attack, and stroke;

Diabetes: normalizes blood sugar, prevents diabetes;

Sex: increases sex drive; and

Anti-aging: reverses tissue “deterioration”, rejuvenates the body.

Safety

7-keto-DHEA is non-toxic. Clinical trials have revealed no safety problems with 7-keto-DHEA used as a supplement at 200 mg/day for 28 days.

Usage

The currently known benefits of 7-keto-DHEA occur at doses in the range of 350 to 1400 mg/day. The supplement is rather expensive at the present time, and its benefits are similar to those of DHEA. 7-keto-DHEA differs from DHEA in its side effects: it does not get converted to estrogens and testosterone in the body, so estrogenic and androgenic side effects are considered to be fewer than for DHEA. These side effects (breast enlargement, acne, growth of body and facial hair) occur only in aminority of users; for those in whom they do, the additional cost may be justified, for others it would not.

Bioavailability and Half-Life

DHEA and other Δ5-androstenes (including 7-keto-DHEA) are extensively metabolized to hydroxylated intermediates in the liver by cytochrome P45family of drug metabolizing enzymes. Ingesting DHEA as an oral supplement requires frequent dosing because much of the hormone is negated during its first pass through the liver. The bioavailability and half-life have not been established for 7-keto-DHEA. As with most other steroidal hormones, micronization improves bioavailability. The presence of other substances that use the same metabolic enzymes can improve bioavailability or half-life, but can also cause side effects.

We have discovered that 7-keto-DHEA may be modified to improve oral bioavailability and plasma half-life in humans and mammals. Specifically, 7-keto-DHEA is modified at the 3^{rd} carbon, the 17^{th} carbon, or at both 3^{rd} carbon and the 17^{th} carbons with one or more of the following: tetrahydropyranyl (both mono and di-ethers), 1-methoxycyclopentane (both mono and di-ethers), cyclopent-1′-enyl (both mono and di-ethers), or combinations thereof. These modifications make the DHEA molecule more lipophilic, permitting circulation through the lymphatic system rather than its normal route. This permits the hormone to induce its desired effect while avoiding rapid metabolism by the liver.

Lymphatic Delivery

But as late as the 1960's and 70's the push was still on to develop effective oral steroids that were not 17-alpha alkylated and did not carry the same unwanted risks of liver toxicity. One concept that was successfully pursued was the notion of bypassing the liver altogether. To do this we need to change the way the steroid is absorbed by the body, so that it will enter circulation through the lymphatic system and not by its normal route. The lymphatic system is responsible for the absorption and distribution of dietary fats, and shuttles these nutrients from the intestines to the lymph nodes so that they can reach peripheral tissues without having to first pass through the liver. To effectively do this however we need to increase the fat solubility of the compound considerably, either by adding a carboxylic acid ester (normally used to create injectable compounds) or an ether group. For our purposes we can look at esters and ethers as essentially the same thing. The key point with both structural additions is that they increase the lipid solubility of the steroid, and therefore the likelihood it will be absorbed by the lymphatic system with dietary fat, yet later break off in circulation (via esterase enzymes) to yield an intact active hormone.

Two lymph-delivered anabolic/androgenic steroids were ultimately developed and marketed by pharmaceutical companies. The first was Anabolicum Vister, which contains boldenone modified with enol ether (quinbolone), and the second Andriol, which uses the undecanoate ester of testosterone. Data is difficult to find on quinbolone, as it was an Italian steroid, however Andriol has been well studied and documented in English text medical journals. The studies are consistent, with Andriol proving to be the only orally effective testosterone product ever developed and commercially sold. Investigators of Andril compared the testosterone response from 100 mg of orally administered testosterone undecanoate, dissolved in oil, with the effects of an equivalent dose (63 mg) of free testosterone. The free testosterone had no noticeable effect on serum levels of testosterone at all, while there was a 2.3 fold increase reported with the single dose of testosterone undecanoate.

This phenomena is utilized in the present invention. 7-keto-DHEA is modified with one of more of the following: tetrahydropyranyl (both mono and di-ethers), 1-methoxycyclopentane (both mono and di-ethers), cyclopent-1′-enyl (both mono and di-ethers), or combinations thereof. These modifications make the DHEA molecule more lipophilic, permitting its entry into the lymphatic system. The hormone avoids being metabolized by the liver and is permitted to induce its desired effect. This permits effective administration of 7-keto-DHEA without frequent and high dosing. Moreover, the modified form may be more cost effective because large quantities need not be administered.

Synthesis

The described modifications of DHEA are readily achieved by those skilled in the art, starting with the parent steroid. The oxygen atoms on the 3^{rd} carbon and the 17^{th} carbons of DHEA readily permit modification through ether linkages.

Among the compounds in the invention are:

Δ 5-Androstene-7alpha-one-3beta, 17beta-diol;

Δ 5-Androstene-3beta,7alpha-diol-17beta-one;

Δ 5-Androstene-7alpha-ol-3beta, 17beta-dione;

Δ 5-Androstene-3beta-acetoxy-7,16-dione-17beta-ol;

Δ 5-Androstene-7alpha, 16alpha-dione-3beta, 17beta-diol;

Δ 5-Androstene-7alpha-one-3beta, 16alpha, 17beta-triol;

Δ 5-Androstene-3beta-propionoxy-16beta-acetoxy-7-one-17beta-ol;

Δ 5-Androstene-7alpha-one-16beta-acetoxy-3beta, 17beta-diol;

Δ 5-Androstene-16alpha-one-3beta,7alpha, 17beta-triol;

Δ 5-Androstene-7alpha-one-3beta, 16alpha,17beta-triol;

Δ 5-Androstene-3beta-ol-7-one-17beta-(1-methoxycyclopentane);

Δ 5-Androstene-7-one-3beta, 17beta-di(1-methoxycyclopentane);

Δ 5-Androstene-7-one-17beta-hydroxy-3-(1-methoxycyclopentane);

Δ 5-Androstene-3beta,7alpha-diol-17beta-(1-methoxycyclopentane);

Δ 5-Androstene-7alpha-ol-3beta, 17beta-di(1-methoxycyclopentane);

Δ 5-Androstene-7alpha, 17beta-dihydroxy-3beta 1-methoxycyclopentane);

Δ 5-Androstene-3beta-ol-7-one-17beta-(1-methoxycyclopentane);

Δ 5-Androstene-7-one-3beta, 17beta-di(1-methoxycyclopentane);

Δ 5-Androstene-17beta-ol-7-one-3beta-(1-methoxycyclopentane);

Δ 5-Androstene-3beta-acetoxy-7,16-dione-17beta-(1-methoxycyclopentane);

Δ 5-Androstene-7,16-dione-3beta, 17beta-di(1-methoxycyclopentane);

Δ 5-Androstene-3beta, 16alpha-dihydroxy-7-one-17beta-(1-methoxycyclopentane);

Δ 5-Androstene-7-one-16alpha-hydroxy-3beta, 17beta-di(1-methoxycyclopentane);

Δ 5-Androstene-3beta-propionoxy-16beta-acetoxy-7-one-17beta-(1-methoxycyclopentane);

Δ 5-Androstene-7-one-16beta-acetoxy-3beta, 17beta-di(1-methoxycyclopentane);

Δ 5-Androstene-3beta,7alpha-diol-16-one-17beta-(1-methoxycyclopentane);

Δ 5-Androstene-7alpha-ol-16-one-3beta, 17beta-di(1-methoxycyclopentane);

Δ 5-Androstene-7alpha, 17beta-dihydroxy-16-one-3beta,-(1-methoxycyclopentane);

Δ 5-Androstene-3beta-ol-7,16-dione-17beta-(1-methoxycyclopentane);

Δ 5-Androstene-17beta-ol-7,16-dione-3beta-(1-methoxycyclopentane);

Δ 5-Androstene-7,16-dione-3beta, 17beta-di(1-methoxycyclopentane);

Δ 5-Androstene-3beta, 16alpha-diol,7-one-17beta-(1-methoxycyclopentane);

Δ 5-Androstene-16alpha-ol,7-one-3beta, 17beta-di(1-methoxycyclopentane);

Δ 5-Androstene-16alpha, 17beta-dihydroxy,7-one-3beta-(1-methoxycyclopentane);

Δ 5-Androstene-3beta-ol-7-one-17beta-cyclopent-1′-enyl;

Δ 5-Androstene-7-one-3beta, 17beta-dicyclopent-1′-enyl;

Δ 5-Androstene-7-one-17beta-hydroxy-3beta-cyclopent-1′-enyl;

Δ 5-Androstene-3beta,7alpha-diol-17beta-cyclopent-1′-enyl;

Δ 5-Androstene-7alpha-ol-3beta, 17beta-dicyclopent-1′-enyl;

Δ 5-Androstene-7alpha, 17beta-dihydroxy-3beta-cyclopent-1′-enyl;

Δ 5-Androstene-3beta-ol-7-one-17beta-cyclopent-1′-enyl;

Δ 5-Androstene-7-one-3beta, 17beta-dicyclopent-1′-enyl;

Δ 5-Androstene-17beta-ol-7-one-3beta-cyclopent-1′-enyl;

Δ 5-Androstene-3beta-acetoxy-7,16-dione-17beta-cyclopent-1′-enyl;

Δ 5-Androstene-7,16-dione-3beta, 17beta-dicyclopent-1′-enyl;

Δ 5-Androstene-3beta, 16alpha-dihydroxy-7-one-17beta-cyclopent-1′-enyl;

Δ 5-Androstene-7-one-16alpha-hydroxy-3beta,17beta-dicyclopent-1′-enyl;

Δ 5-Androstene-3beta-propionoxy-16beta-acetoxy-7-one-17beta-cyclopent-1′-enyl;

Δ 5-Androstene-7-one-16beta-acetoxy-3beta, 17beta-dicyclopent-1′-enyl;

Δ 5-Androstene-3beta,7alpha-diol-16-one-17beta-cyclopent-1′-enyl;

Δ 5-Androstene-7alpha-ol-16-one-3beta,17beta-dicyclopent-1′-enyl;

Δ 5-Androstene-7alpha, 17beta-dihydroxy-16-one-3beta-cyclopent-1′-enyl;

Δ 5-Androstene-3beta-ol-7,16-dione-17beta-cyclopent-1′-enyl;

Δ 5-Androstene-7,16-dione-3beta, 17beta-dicyclopent-1′-enyl;

Δ 5-Androstene-17beta-ol-7,16-dione-3beta-cyclopent-1′-enyl;

Δ 5-Androstene-3beta, 16alpha-diol,7-one-17beta-cyclopent-1′-enyl;

Δ 5-Androstene-16alpha-ol,7-one-3beta, 17beta-dicyclopent-1′-enyl;

Δ 5-Androstene-16alpha, 17beta-diol,7-one-3beta-cyclopent-1′-enyl;

Δ 5-Androstene-3beta-ol-7-one-17beta-tetrahydropyranyl;

Δ 5-Androstene-7-one-3beta,17beta-ditetrahydropyranyl;

Δ 5-Androstene-17beta-ol-7-one-3beta-tetrahydropyranyl;

Δ 5-Androstene-3beta,7alpha-diol-17beta-tetrahydropyranyl;

Δ 5-Androstene-7alpha-ol-3beta, 17beta-ditetrahydropyranyl;

Δ 5-Androstene-3beta-ol-7-one-17beta-tetrahydropyranyl;

Δ 5-Androstene-7-one-3beta,17beta-ditetrahydropyranyl;

Δ 5-Androstene-17beta-ol-7-one-3beta-tetrahydropyranyl;

Δ 5-Androstene-3beta-acetoxy-7,16-dione-17beta-tetrahydropyranyl;

Δ 5-Androstene-7,16-dione-3beta, 17beta-ditetrahydropyranyl;

Δ 5-Androstene-3beta 16alpha-dihydroxy-7-one-17beta-tetrahydropyranyl;

Δ 5-Androstene-7-one-16alpha-hydroxy-3beta,17beta-ditetrahydropyranyl;

Δ 5-Androstene-16alpha,17beta-dihydroxy-7-one-3beta-tetrahydropyranyl;

Δ 5-Androstene-3beta-propionoxy-16beta-acetoxy-7-one-17beta-tetrahydropyranyl;

Δ 5-Androstene-7-one-16beta-acetoxy-3beta, 17beta-ditetrahydropyranyl;

Δ 5-Androstene-3beta,7alpha-diol-16-one-17beta-tetrahydropyranyl;

Δ 5-Androstene-7alpha-ol-16-one-3beta, 17beta-ditetrahydropyranyl;

Δ 5-Androstene-7alpha, 17beta-diol-16-one-3beta-tetrahydropyranyl;

Δ 5-Androstene-3beta-ol-7,16-dione-17beta-tetrahydropyranyl;

Δ 5-Androstene-7,16-dione-3beta, 17beta-ditetrahydropyranyl;

Δ 5-Androstene-17beta-ol-7,16-dione-3beta-tetrahydropyranyl;

Δ 5-Androstene-3beta, 16alpha-diol,7-one-17beta-tetrahydropyranyl;

Δ 5-Androstene-16alpha-ol,7-one-3beta, 17beta-ditetrahydropyranyl; and

Δ 5-Androstene-6alpha, 17beta-diol,7-one-3beta-tetrahydropyranyl.

With the compounds of the present invention a substantial increase in oral bioavailability and plasma half life in mammals as compared to non-modified Δ5-androstene compounds. Furthermore, advantageous treatment with one or more compounds of the present invention will improve a subject's memory, improve neurological health, improve weight loss by increasing fat reduction, increase energy, reduce fatigue, improve immune response, increase T3 thyroid hormone activity, and combinations thereof.

Although the invention has been described with reference to one or more preferred embodiments, this description is not to be construed in a limiting sense. There is modification of the disclosed embodiments, as well as alternative embodiments of this invention, which will be apparent to persons of ordinary skill in the art, and the invention shall be viewed as limited only by reference to the following claims.

What is claimed is:

1. A hormone for eliciting a desired response comprising at least one modified Δ5-androstene compound.

2. The hormone according to claim 1, wherein said hormone is modified at the 3^{rd} carbon with one or more of the following: tetrahydropyranyl (both mono and di-ethers), 1-methoxycyclopentane (both mono and di-ethers), cyclopent-1′-enyl (both mono and di-ethers), or combinations thereof.

3. The hormone according to claim 1, wherein said hormone is modified at the 17^{th} carbon with at least one of the following: tetrahydropyranyl (both mono and di-ethers), 1-methoxycyclopentane (both mono and di-ethers), cyclopent-1′-enyl (both mono and di-ethers), or combinations thereof.

4. The hormone according to claim 1, wherein said hormone is modified at both the 3^{rd} and 17^{th} carbons with at least one of the following: tetrahydropyranyl (both mono and di-ethers), 1-methoxycyclopentane (both mono and di-ethers), cyclopent-1′-enyl (both mono and di-ethers), or combinations thereof.

5. The hormone according to claim 1 wherein the at least one modified Δ5-androstene compound is selected from the group consisting of
Δ 5-Androstene-7alpha-one-3beta, 17beta-diol;
Δ 5-Androstene-3beta,7alpha-diol-17beta-one;
Δ 5-Androstene-7alpha-ol-3beta, 17beta-dione;
Δ 5-Androstene-3beta-acetoxy-7,16-dione-17beta-ol;
Δ 5-Androstene-7alpha, 16alpha-dione-3beta, 17beta-diol;
Δ 5-Androstene-7alpha-one-3beta, 16alpha,17beta-triol;
Δ 5-Androstene-3beta-propionoxy-16beta-acetoxy-7-one-17beta-ol;
Δ 5-Androstene-7alpha-one-16beta-acetoxy-3beta, 17beta-diol;
Δ 5-Androstene-16alpha-one-3beta,7alpha, 17beta-triol;
Δ 5-Androstene-7alpha-one-3beta, 16alpha,17beta-triol;
Δ 5-Androstene-3beta-ol-7-one-17beta-(1-methoxycyclopentane);
Δ 5-Androstene-7-one-3beta, 17beta-di(1-methoxycyclopentane);
Δ 5-Androstene-7-one-17beta-hydroxy-3-(1-methoxycyclopentane);
Δ 5-Androstene-3beta,7alpha-diol-17beta-(1-methoxycyclopentane);
Δ 5-Androstene-7alpha-ol-3beta, 17beta-di(1-methoxycyclopentane);
Δ 5-Androstene-7alpha, 17beta-dihydroxy-3beta-(1-methoxycyclopentane);
Δ 5-Androstene-3beta-ol-7-one-17beta-(1-methoxycyclopentane);
Δ 5-Androstene-7-one-3beta, 17beta-di(1-methoxycyclopentane);
Δ 5-Androstene-17beta-ol-7-one-3beta-(1-methoxycyclopentane);
Δ 5-Androstene-3beta-acetoxy-7,16-dione-17beta-(1-methoxycyclopentane);
Δ 5-Androstene-7,16-dione-3beta, 17beta-di(1-methoxycyclopentane);
Δ 5-Androstene-3beta, 16alpha-dihydroxy-7-one-17beta-(1-methoxycyclopentane);
Δ 5-Androstene-7-one-16alpha-hydroxy-3beta, 17beta-di(1-methoxycyclopentane);
Δ 5-Androstene-3beta-propionoxy-16beta-acetoxy-7-one-17beta-(1-methoxycyclopentane);
Δ 5-Androstene-7-one-16beta-acetoxy-3beta, 17beta-di(1-methoxycyclopentane);
Δ 5-Androstene-3beta,7alpha-diol-16-one-17beta-(1-methoxycyclopentane);
Δ 5-Androstene-7alpha-ol-16-one-3beta, 17beta-di(1-methoxycyclopentane);
Δ 5-Androstene-7alpha, 17beta-dihydroxy-16-one-3beta,-(1-methoxycyclopentane);
Δ 5-Androstene-3beta-ol-7,16-dione-17beta-(1-methoxycyclopentane);
Δ 5-Androstene-17beta-ol-7,16-dione-3beta-(1-methoxycyclopentane);
Δ 5-Androstene-7,16-dione-3beta, 17beta-di(1-methoxycyclopentane);
Δ 5-Androstene-3beta, 16alpha-diol,7-one-17beta-(1-methoxycyclopentane);
Δ 5-Androstene-16alpha-ol,7-one-3beta, 17beta-di(1-methoxycyclopentane);
Δ 5-Androstene-16alpha, 17beta-dihydroxy,7-one-3beta-(1-methoxycyclopentane);
Δ 5-Androstene-3beta-ol-7-one-17beta-cyclopent-1′-enyl;
Δ 5-Androstene-7-one-3beta, 17beta-dicyclopent-1′-enyl;
Δ 5-Androstene-7-one-17beta-hydroxy-3beta-cyclopent-1′-enyl
Δ 5-Androstene-3beta,7alpha-diol-17beta-cyclopent-1′-enyl;
Δ 5-Androstene-7alpha-ol-3beta, 17beta-dicyclopent-1′-enyl;
Δ 5-Androstene-7alpha, 17beta-dihydroxy-3beta-cyclopent-1′-enyl;
Δ 5-Androstene-3beta-ol-7-one-17beta-cyclopent-1′-enyl;
Δ 5-Androstene-7-one-3beta, 17beta-dicyclopent-1′-enyl;
Δ 5-Androstene-17beta-ol-7-one-3beta-cyclopent-1′-enyl;
Δ 5-Androstene-3beta-acetoxy-7,16-dione-17beta-cyclopent-1′-enyl;
Δ 5-Androstene-7,16-dione-3beta, 17beta-dicyclopent-1′-enyl;
Δ 5-Androstene-3beta, 16alpha-dihydroxy-7-one-17beta-cyclopent-1′-enyl;
Δ 5-Androstene-7-one-16alpha-hydroxy-3beta, 17beta-dicyclopent-1′-enyl;
Δ 5-Androstene-3beta-propionoxy-16beta-acetoxy-7-one-17beta-cyclopent-1′-enyl;
Δ 5-Androstene-7-one-16beta-acetoxy-3beta, 17beta-dicyclopent-1′-enyl;
Δ 5-Androstene-3beta,7alpha-diol-16-one-17beta-cyclopent-1′-enyl;
Δ 5-Androstene-7alpha-ol-16-one-3beta, 17beta-dicyclopent-1′-enyl;
Δ 5-Androstene-7alpha, 17beta-dihydroxy-16-one-3beta-cyclopent-11′-enyl;
Δ 5-Androstene-3beta-ol-7,16-dione-17beta-cyclopent-1′-enyl;
Δ 5-Androstene-7,16-dione-3beta, 17beta-dicyclopent-1′-enyl;
Δ 5-Androstene-17beta-ol-7,16-dione-3beta-cyclopent-1′-enyl;
Δ 5-Androstene-3beta, 16alpha-diol,7-one-17beta-cyclopent-1′-enyl;
Δ 5-Androstene-16alpha-ol,7-one-3beta, 17beta-dicyclopent-1′-enyl;
Δ 5-Androstene-16alpha, 17beta-diol,7-one-3beta-cyclopent-1′-enyl;
Δ 5-Androstene-3beta-ol-7-one-17beta-tetrahydropyranyl;
Δ 5-Androstene-7-one-3beta, 17beta-ditetrahydropyranyl;
Δ 5-Androstene-17beta-ol-7-one-3beta-tetrahydropyranyl;
Δ 5-Androstene-3beta,7alpha-diol-17beta-tetrahydropyranyl;
Δ 5-Androstene-7alpha-ol-3beta, 17beta-ditetrahydropyranyl;
Δ 5-Androstene-3beta-ol-7-one-17beta-tetrahydropyranyl;
Δ 5-Androstene-7-one-3beta, 17beta-ditetrahydropyranyl;
Δ 5-Androstene-17beta-ol-7-one-3beta-tetrahydropyranyl;
Δ 5-Androstene-3beta-acetoxy-7,16-dione-17beta-tetrahydropyranyl;
Δ 5-Androstene-7,16-dione-3beta, 17beta-ditetrahydropyranyl;
Δ 5-Androstene-3beta, 16alpha-dihydroxy-7-one-17beta-tetrahydropyranyl;
Δ 5-Androstene-7-one-16alpha-hydroxy-3beta, 17beta-ditetrahydropyranyl;
Δ 5-Androstene-16alpha, 17beta-dihydroxy-7-one-3beta-tetrahydropyranyl;
Δ 5-Androstene-3beta-propionoxy-16beta-acetoxy-7-one-17beta-tetrahydropyranyl;
Δ 5-Androstene-7-one-16beta-acetoxy-3beta, 17beta-ditetrahydropyranyl;
Δ 5-Androstene-3beta,7alpha-diol-16-one-17beta-tetrahydropyranyl;
Δ 5-Androstene-7alpha-ol-16-one-3beta, 17beta-ditetrahydropyranyl;
Δ 5-Androstene-7alpha, 17beta-diol-16-one-3beta-tetrahydropyranyl;
Δ 5-Androstene-3beta-ol-7,16-dione-17beta-tetrahydropyranyl;
Δ 5-Androstene-7,16-dione-3beta, 17beta-ditetrahydropyranyl;
Δ 5-Androstene-17beta-ol-7,16-dione-3beta-tetrahydropyranyl;
Δ 5-Androstene-3beta, 16alpha-diol,7-one-17beta-tetrahydropyranyl;
Δ 5-Androstene-16alpha-ol,7-one-3beta, 17beta-ditetrahydropyranyl; and
Δ 5-Androstene-6alpha, 17beta-diol,7-one-3beta-tetrahydropyranyl.

6. The hormone of claim 5 wherein the modification produces a substantial increase in oral bioavailability and plasma half life in mammals as compared to non-modified Δ5-androstene compounds.

7. A method for using a hormone to elicit a desired response comprising: administering an effective amount of a supplement having at least one modified Δ5-androstene compounds selected from the group consisting of
Δ 5-Androstene-7alpha-one-3beta, 17beta-diol;
Δ 5-Androstene-3beta,7alpha-diol-17beta-one;
Δ 5-Androstene-7alpha-ol-3beta, 17beta-dione;
Δ 5-Androstene-3beta-acetoxy-7,16-dione-17beta-ol;
Δ 5-Androstene-7alpha,16alpha-dione-3beta, 17beta-diol;
Δ 5-Androstene-7alpha-one-3beta, 16alpha, 17beta-triol;
Δ 5-Androstene-3beta-propionoxy-16beta-acetoxy-7-one-17beta-ol;
Δ 5-Androstene-7alpha-one-16beta-acetoxy-3beta, 17beta-diol;
Δ 5-Androstene-16alpha-one-3beta,7alpha, 17beta-triol;
Δ 5-Androstene-7alpha-one-3beta, 16alpha,17beta-triol;
Δ 5-Androstene-3beta-ol-7-one-17beta-(1-methoxycyclopentane);
Δ 5-Androstene-7-one-3beta,17beta-di(1-methoxycyclopentane);
Δ 5-Androstene-7-one-17beta-hydroxy-3-(1-methoxycyclopentane);
Δ 5-Androstene-3beta,7alpha-diol-17beta-(1-methoxycyclopentane);
Δ 5-Androstene-7alpha-ol-3beta, 17beta-di(1-methoxycyclopentane);
Δ 5-Androstene-7alpha, 17beta-dihydroxy-3beta-(1-methoxycyclopentane);
Δ 5-Androstene-3beta-ol-7-one-17beta-(1-methoxycyclopentane);
Δ 5-Androstene-7-one-3beta, 17beta-di(1-methoxycyclopentane);
Δ 5-Androstene-17beta-ol-7-one-3beta-(1-methoxycyclopentane);
Δ 5-Androstene-3beta-acetoxy-7,16-dione-17beta-(1-methoxycyclopentane);
Δ 5-Androstene-7,16-dione-3beta, 17beta-di(1-methoxycyclopentane);
Δ 5-Androstene-3beta, 16alpha-dihydroxy-7-one-17beta-(1-methoxycyclopentane);
Δ 5-Androstene-7-one-16alpha-hydroxy-3beta, 17beta-di(1-methoxycyclopentane);
Δ 5-Androstene-3beta-propionoxy-16beta-acetoxy-7-one-17beta-(1-methoxycyclopentane);
Δ 5-Androstene-7-one-16beta-acetoxy-3beta,17beta-di(1-methoxycyclopentane);
Δ 5-Androstene-3beta,7alpha-diol-16-one-17beta-(1-methoxycyclopentane);
Δ 5-Androstene-7alpha-ol-16-one-3beta, 17beta-di(1-methoxycyclopentane);
Δ 5-Androstene-7alpha, 17beta-dihydroxy-16-one-3beta,-(1-methoxycyclopentane);
Δ 5-Androstene-3beta-ol-7,16-dione-17beta-(1-methoxycyclopentane);
Δ 5-Androstene-17beta-ol-7,16-dione-3beta-(1-methoxycyclopentane);
Δ 5-Androstene-7,16-dione-3beta, 17beta-di(1-methoxycyclopentane);
Δ 5-Androstene-3beta, 16alpha-diol,7-one-17beta-(1-methoxycyclopentane);
Δ 5-Androstene-16alpha-ol,7-one-3beta, 17beta-di(1-methoxycyclopentane);
a 5-Androstene-16alpha, 17beta-dihydroxy,7-one-3beta-(1-methoxycyclopentane);
Δ 5-Androstene-3beta-ol-7-one-17beta-cyclopent-1′-enyl;
Δ 5-Androstene-7-one-3beta,17beta-dicyclopent-1′-enyl;
Δ 5-Androstene-7-one-17beta-hydroxy-3beta-cyclopent-1′-enyl
Δ 5-Androstene-3beta,7alpha-diol-17beta-cyclopent-1′-enyl;
Δ 5-Androstene-7alpha-ol-3beta, 17beta-dicyclopent-1′-enyl;
Δ 5-Androstene-7alpha, 17beta-dihydroxy-3beta-cyclopent-1′-enyl;
Δ 5-Androstene-3beta-ol-7-one-17beta-cyclopent-1′-enyl;
Δ 5-Androstene-7-one-3beta, 17beta-dicyclopent-1′-enyl;
Δ 5-Androstene-17beta-ol-7-one-3beta-cyclopent-1′-enyl;
Δ 5-Androstene-3beta-acetoxy-7,16-dione-17beta-cyclopent-1′-enyl;
Δ 5-Androstene-7,16-dione-3beta, 17beta-dicyclopent-1′-enyl;
Δ 5-Androstene-3beta, 16alpha-dihydroxy-7-one-17beta-cyclopent-1′-enyl;
Δ 5-Androstene-7-one-16alpha-hydroxy-3beta, 17beta-dicyclopent-1′-enyl;
Δ 5-Androstene-3beta-propionoxy-16beta-acetoxy-7-one-17beta-cyclopent-1′-enyl;
Δ 5-Androstene-7-one-16beta-acetoxy-3beta, 17beta-dicyclopent-1′-enyl;
Δ 5-Androstene-3beta,7alpha-diol-16-one-17beta-cyclopent-1′-enyl;
Δ 5-Androstene-7alpha-ol-16-one-3beta, 17beta-dicyclopent-1′-enyl;
Δ 5-Androstene-7alpha, 17beta-dihydroxy-16-one-3beta-cyclopent-1′-enyl;
Δ 5-Androstene-3beta-ol-7,16-dione-17beta-cyclopent-1′-enyl;
Δ 5-Androstene-7,16-dione-3beta, 17beta-dicyclopent-1′-enyl;
Δ 5-Androstene-17beta-ol-7,16-dione-3beta-cyclopent-1′-enyl;
Δ 5-Androstene-3beta, 16alpha-diol,7-one-17beta-cyclopent-1′-enyl;
Δ 5-Androstene-16alpha-ol,7-one-3beta, 17beta-dicyclopent-1′-enyl;
Δ 5-Androstene-16alpha, 17beta-diol,7-one-3beta-cyclopent-1′-enyl;
Δ 5-Androstene-3beta-ol-7-one-17beta-tetrahydropyranyl;
Δ 5-Androstene-7-one-3beta,17beta-ditetrahydropyranyl;
Δ 5-Androstene-17beta-ol-7-one-3beta-tetrahydropyranyl;
Δ 5-Androstene-3beta,7alpha-diol-17beta-tetrahydropyranyl;
Δ 5-Androstene-7alpha-ol-3beta, 17beta-ditetrahydropyranyl;
Δ 5-Androstene-3beta-ol-7-one-17beta-tetrahydropyranyl;
Δ 5-Androstene-7-one-3beta, 17beta-ditetrahydropyranyl;
Δ 5-Androstene-17beta-ol-7-one-3beta-tetrahydropyranyl;
Δ 5-Androstene-3beta-acetoxy-7,16-dione-17beta-tetrahydropyranyl;
Δ 5-Androstene-7,16-dione-3beta, 17beta-ditetrahydropyranyl;
Δ 5-Androstene-3beta, 16alpha-dihydroxy-7-one-17beta-tetrahydropyranyl;
Δ 5-Androstene-7-one-16alpha-hydroxy-3beta, 17beta-ditetrahydropyranyl;
Δ 5-Androstene-16alpha, 17beta-dihydroxy-7-one-3beta-tetrahydropyranyl;
Δ 5-Androstene-3beta-propionoxy-16beta-acetoxy-7-one-17beta-tetrahydropyranyl;
Δ 5-Androstene-7-one-16beta-acetoxy-3beta, 17beta-ditetrahydropyranyl;
Δ 5-Androstene-3beta,7alpha-diol-16-one-17beta-tetrahydropyranyl;
Δ 5-Androstene-7alpha-ol-16-one-3beta, 17beta-ditetrahydropyranyl;
Δ 5-Androstene-7alpha-17-beta-diol-16-one-3beta-tetrahydropyranyl;
Δ 5-Androstene-3beta-ol-7,16-dione-17beta-tetrahydropyranyl;
Δ 5-Androstene-7,16-dione-3beta, 17beta-ditetrahydropyranyl;
Δ 5-Androstene-17beta-ol-7,16-dione-3beta-tetrahydropyranyl;
Δ 5-Androstene-3beta, 16alpha-diol,7-one-17beta-tetrahydropyranyl;
Δ 5-Androstene-16alpha-ol,7-one-3beta,17beta-ditetrahydropyranyl; and
Δ 5-Androstene-6alpha,17beta-diol,7-one-3beta-tetrahydropyranyl.

8. The method according to claim 7 wherein the desired response comprises at least one of the following: improving weight loss by increasing fat reduction, increasing energy, reducing fatigue, increasing T3 thyroid hormone activity, or combinations thereof.

9. The method according to claim 7 wherein said hormone is modified at the 3^{rd} carbon with at least one of the following: tetrahydropyranyl (both mono and di-ethers), 1-methoxycyclopentane (both mono and di-ethers), cyclopent-1′-enyl (both mono and di-ethers), or combinations thereof.

10. The method according to claim 7 wherein said hormone is modified at the 17^{th} carbon with at least one of the following: tetrahydropyranyl (both mono and di-ethers), 1-methoxycyclopentane (both mono and di-ethers), cyclopent-1′-enyl (both mono and di-ethers), or combinations thereof.

11. The method according to claim 7 wherein said hormone is modified at both the 3^{rd} and 17^{th} carbons with at least one of the following: tetrahydropyranyl (both mono and di-ethers), 1-methoxycyclopentane (both mono and di-ethers), cyclopent-1′-enyl (both mono and di-ethers), or combinations thereof.

12. The method of claim 7 wherein the desired response includes an improved immune response, improved neurological condition, improved memory, increased fat reduction, increased energy or combinations thereof.

13. The method of claim 7 wherein the at least one modified Δ5-androstene compounds produces a substantial increase in oral bioavailability and plasma half-life in mammals as compared to non-modified Δ5-androstene compounds.

14. The method of claim 8 wherein the at least one modified Δ5-androstene compounds produces a substantial increase in oral bioavailability and plasma half-life in mammals as compared to non-modified Δ5-androstene compounds.

15. A method for eliciting a desired response using a hormone comprising: administering an effective amount of compound selected from the group consisting of
Δ 5-Androstene-7alpha-one-3beta, 17beta-diol;
Δ 5-Androstene-3beta,7alpha-diol-17beta-one;
Δ 5-Androstene-7alpha-ol-3beta, 17beta-dione;
Δ 5-Androstene-3beta-acetoxy-7,16-dione-17beta-ol;
Δ 5-Androstene-7alpha, 16alpha-dione-3beta, 17beta-diol;
Δ 5-Androstene-7alpha-one-3beta, 16alpha, 17beta-triol;
Δ 5-Androstene-3beta-propionoxy-16beta-acetoxy-7-one-17beta-ol;
Δ 5-Androstene-7alpha-one-16beta-acetoxy-3beta, 17beta-diol;
Δ 5-Androstene-16alpha-one-3beta,7alpha, 17beta-triol;
Δ 5-Androstene-7alpha-one-3beta, 16alpha,17beta-triol;
Δ 5-Androstene-3beta-ol-7-one-17beta-(1-methoxycyclopentane);
Δ 5-Androstene-7-one-3beta, 17beta-di(1-methoxycyclopentane);
Δ 5-Androstene-7-one-17beta-hydroxy-3-(1-methoxycyclopentane);
Δ 5-Androstene-3beta,7alpha-diol-17beta-(1-methoxycyclopentane);
Δ 5-Androstene-7alpha-ol-3 beta, 17beta-di(1-methoxycyclopentane);
Δ 5-Androstene-7alpha, 17beta-dihydroxy-3beta-(1-methoxycyclopentane);
Δ 5-Androstene-3beta-ol-7-one-17beta-(1-methoxycyclopentane);
Δ 5-Androstene-7-one-3beta, 17beta-di(1-methoxycyclopentane);
Δ 5-Androstene-17beta-ol-7-one-3beta-(1-methoxycyclopentane);
Δ 5-Androstene-3beta-acetoxy-7,16-dione-17beta-(1-methoxycyclopentane);
Δ 5-Androstene-7,16-dione-3beta, 17beta-di(1-methoxycyclopentane);
Δ 5-Androstene-3beta, 16alpha-dihydroxy-7-one-17beta-(1-methoxycyclopentane);
Δ 5-Androstene-7-one-16alpha-hydroxy-3beta, 17beta-di(1-methoxycyclopentane);
Δ 5-Androstene-3beta-propionoxy-16beta-acetoxy-7-one-17beta-(1-methoxycyclopentane);
Δ 5-Androstene-7-one-16beta-acetoxy-3beta, 17beta-di(1-methoxycyclopentane);
Δ 5-Androstene-3beta,7alpha-diol-16-one-17beta-(1-methoxycyclopentane);
Δ 5-Androstene-7alpha-ol-16-one-3beta, 17beta-di(1-methoxycyclopentane);
Δ 5-Androstene-7alpha, 17beta-dihydroxy-16-one-3beta,-(1-methoxycyclopentane);
Δ 5-Androstene-3beta-ol-7,16-dione-17beta-(1-methoxycyclopentane);
Δ 5-Androstene-17beta-ol-7,16-dione-3beta-(1-methoxycyclopentane);
Δ 5-Androstene-7,16-dione-3beta, 17beta-di(1-methoxycyclopentane);
Δ 5-Androstene-3beta, 16alpha-diol,7-one-17beta-(1-methoxycyclopentane);
Δ 5-Androstene-16alpha-ol,7-one-3beta, 17beta-di(1-methoxycyclopentane);
Δ 5-Androstene-16alpha, 17beta-dihydroxy,7-one-3beta-(1-methoxycyclopentane);
Δ 5-Androstene-3beta-ol-7-one-17beta-cyclopent-1′-enyl;
Δ 5-Androstene-7-one-3beta, 17beta-dicyclopent-1′-enyl;
Δ 5-Androstene-7-one-17beta-hydroxy-3beta-cyclopent-1′-enyl;
Δ 5-Androstene-3beta,7alpha-diol-17beta-cyclopent-1′-enyl;
Δ 5-Androstene-7alpha-ol-3beta, 17beta-dicyclopent-1′-enyl;
Δ 5-Androstene-7alpha, 17beta-dihydroxy-3beta-cyclopent-1′-enyl;
Δ 5-Androstene-3beta-ol-7-one-17beta-cyclopent-1′-enyl;
Δ 5-Androstene-7-one-3beta, 17beta-dicyclopent-1′-enyl;
Δ 5-Androstene-17beta-ol-7-one-3beta-cyclopent-1′-enyl;
Δ 5-Androstene-3beta-acetoxy-7,16-dione-17beta-cyclopent-1′-enyl;
Δ 5-Androstene-7,16-dione-3beta, 17beta-dicyclopent-1′-enyl;
Δ 5-Androstene-3beta, 16alpha-dihydroxy-7-one-17beta-cyclopent-1′-enyl;
Δ 5-Androstene-7-one-16alpha-hydroxy-3beta, 17beta-dicyclopent-1′-enyl;
Δ 5-Androstene-3beta-propionoxy-16beta-acetoxy-7-one-17beta-cyclopent-1′-enyl;
Δ 5-Androstene-7-one-16beta-acetoxy-3beta, 17beta-dicyclopent-1′-enyl;
Δ 5-Androstene-3beta,7alpha-diol-16-one-17beta-cyclopent-1′-enyl;
Δ 5-Androstene-7alpha-ol-16-one-3beta, 17beta-dicyclopent-1′-enyl;
Δ 5-Androstene-7alpha, 17beta-dihydroxy-16-one-3beta-cyclopent-1′-enyl;
Δ 5-Androstene-3beta-ol-7,16-dione-17beta-cyclopent-1′-enyl;
Δ 5-Androstene-7,16-dione-3beta, 17beta-dicyclopent-1′-enyl;
Δ 5-Androstene-17beta-ol-7,16-dione-3beta-cyclopent-1′-enyl;
Δ 5-Androstene-3beta, 16alpha-diol,7-one-17beta-cyclopent-1′-enyl;
Δ 5-Androstene-16alpha-ol,7-one-3beta, 17beta-dicyclopent-1′-enyl;
Δ 5-Androstene-16alpha, 17beta-diol,7-one-3beta-cyclopent-1′-enyl;
Δ 5-Androstene-3beta-ol-7-one-17beta-tetrahydropyranyl;
Δ 5-Androstene-7-one-3beta, 17beta-ditetrahydropyranyl;
Δ 5-Androstene-17beta-ol-7-one-3beta-tetrahydropyranyl;
Δ 5-Androstene-3beta,7alpha-diol-17beta-tetrahydropyranyl;
Δ 5-Androstene-7alpha-ol-3beta, 17beta-ditetrahydropyranyl;
Δ 5-Androstene-3beta-ol-7-one-17beta-tetrahydropyranyl;
Δ 5-Androstene-7-one-3beta, 17beta-ditetrahydropyranyl;
Δ 5-Androstene-17beta-ol-7-one-3beta-tetrahydropyranyl;
Δ 5-Androstene-3beta-acetoxy-7,16-dione-17beta-tetrahydropyranyl;
Δ 5-Androstene-7,16-dione-3beta, 17beta-ditetrahydropyranyl;
Δ 5-Androstene-3beta, 16alpha-dihydroxy-7-one-17beta-tetrahydropyranyl;
Δ 5-Androstene-7-one-16alpha-hydroxy-3beta, 17beta-ditetrahydropyranyl;
Δ 5-Androstene-16alpha, 17beta-dihydroxy-7-one-3beta-tetrahydropyranyl;
Δ 5-Androstene-3beta-propionoxy-16beta-acetoxy-7-one-17beta-tetrahydropyranyl;
Δ 5-Androstene-7-one-16beta-acetoxy-3 beta, 17beta-ditetrahydropyranyl;
Δ 5-Androstene-3beta,7alpha-diol-16-one-17beta-tetrahydropyranyl;
Δ 5-Androstene-7alpha-ol-16-one-3beta, 17beta-ditetrahydropyranyl;
Δ 5-Androstene-7alpha, 17beta-diol-16-one-3beta-tetrahydropyranyl;
Δ 5-Androstene-3beta-ol-7,16-dione-17beta-tetrahydropyranyl;
Δ 5-Androstene-7,16-dione-3beta, 17beta-ditetrahydropyranyl;
Δ 5-Androstene-17beta-ol-7,16-dione-3beta-tetrahydropyranyl;
Δ 5-Androstene-3beta, 16alpha-diol,7-one-17beta-tetrahydropyranyl;
Δ 5-Androstene-16alpha-ol,7-one-3beta, 17beta-ditetrahydropyranyl; and
Δ 5-Androstene-6alpha, 17beta-diol,7-one-3beta-tetrahydropyranyl.

16. The method of claim 15 wherein the desired response includes an improved immune response, improved neurological condition, improved memory, or combinations thereof.

17. The method of claim 15 wherein the desired response includes increased fat reduction, increased energy, reduced fatigue or combinations thereof.

18. The method of claim 15 wherein the desired response includes an improvement in symptoms of one or more of the following ailments: HIV/Aids, heart disease, lupus, diabetes, depression, low sex drive, and muscular wasting disease.

19. The method of claim 15 wherein the desired response includes increased T3 thyroid hormone activity.

20. The method of claim 15 wherein the compound produces a substantial increase in oral bioavailability and plasma half life in mammals as compared to non-modified Δ5-androstene compounds.
